(19) United States
(12) Patent Application Publication
(10) Pub. No.: US 2021/0378643 A1
(43) Pub. Date: Dec. 9, 2021

(54) BREAKABLE SAMPLE COLLECTION SWAB

(71) Applicant: Detect, Inc., Guilford, CT (US)

(72) Inventors: Todd Roswech, Ivoryton, CT (US); Jonathan M. Rothberg, Guilford, CT (US); Spencer Glantz, West Hartford, CT (US); Benjamin Rosenbluth, Hamden, CT (US)

(73) Assignee: Detect, Inc., Guilford, CT (US)

(21) Appl. No.: 17/338,469

(22) Filed: Jun. 3, 2021

Related U.S. Application Data
(60) Provisional application No. 63/161,607, filed on Mar. 16, 2021.
(60) Provisional application No. 63/148,250, filed on Feb. 11, 2021.
(60) Provisional application No. 63/110,783, filed on Nov. 6, 2020.
(60) Provisional application No. 63/089,801, filed on Oct. 9, 2020.
(60) Provisional application No. 63/086,196, filed on Oct. 1, 2020.
(60) Provisional application No. 63/081,201, filed on Sep. 21, 2020.
(60) Provisional application No. 63/068,303, filed on Aug. 20, 2020.
(60) Provisional application No. 63/066,770, filed on Aug. 17, 2020.
(60) Provisional application No. 63/066,111, filed on Aug. 14, 2020.
(60) Provisional application No. 63/065,131, filed on Aug. 13, 2020.
(60) Provisional application No. 63/063,931, filed on Aug. 10, 2020.
(60) Provisional application No. 63/061,072, filed on Aug. 4, 2020.
(60) Provisional application No. 63/059,928, filed on Jul. 31, 2020.
(60) Provisional application No. 63/053,534, filed on Jul. 17, 2020.
(60) Provisional application No. 63/034,901, filed on Jun. 4, 2020.

(51) Int. Cl. A61B 10/00; B01L 3/00; C12Q 1/6844; C12Q 1/70

(52) U.S. Cl. CPC A61B 10/0051; C12Q 1/70; C12Q 1/6844; B01L 3/5029

(57) ABSTRACT

Provided herein, in some embodiments, are breakable swabs for sample collection. In some embodiments, the swabs may be used in rapid diagnostic tests to detect one or more target nucleic acid sequences (e.g., a nucleic acid sequence of one or more pathogens). In some embodiments, the pathogens are viral, bacterial, fungal, parasitic, or protozoan pathogens, such as SARS-CoV-2 or an influenza virus. The swab may include a stem portion for handling and a head portion for collecting and/or holding a sample. After sample collection, the head portion may be inserted into a reaction tube or any other sample-receiving device. The stem portion of the swab may then be separated or detached from the head portion along a breakable portion.

### CROSS-REFERENCE TO RELATED APPLICATIONS

This application claims the benefit under 35 U.S.C. § 119(e) of U.S. Provisional Application Ser. No. 63/161,607, filed Mar. 16, 2021, U.S. Provisional Application Ser. No. 63/148,250, filed Feb. 11, 2021, U.S. Provisional Application Ser. No. 63/110,783, filed Nov. 6, 2020, U.S. Provisional Application Ser. No. 63/089,801, filed Oct. 9, 2020, U.S. Provisional Application Ser. No. 63/086,196, filed Oct. 1, 2020, U.S. Provisional Application Ser. No. 63/081,201, filed Sep. 21, 2020, U.S. Provisional Application Ser. No. 63/068,303, filed Aug. 20, 2020, U.S. Provisional Application Ser. No. 63/066,770, filed Aug. 17, 2020, U.S. Provisional Application Ser. No. 63/066,111, filed Aug. 14, 2020, U.S. Provisional Application Ser. No. 63/065,131, filed Aug. 13, 2020, U.S. Provisional Application Ser. No. 63/063,931, filed Aug. 10, 2020, U.S. Provisional Application Ser. No. 63/061,072, filed Aug. 4, 2020, U.S. Provisional Application Ser. No. 63/059,928, filed Jul. 31, 2020, U.S. Provisional Application Ser. No. 63/053,534, filed Jul. 17, 2020, and U.S. Provisional Application Ser. No. 63/034,901, filed Jun. 4, 2020, each of which is hereby incorporated by reference in its entirety.

### FIELD

The present invention generally relates to diagnostic devices, systems, and methods for detecting the presence of a target nucleic acid sequence.

### BACKGROUND

The ability to rapidly diagnose diseases—particularly highly infectious diseases—is critical to preserving human health. Viral infections, such as coronaviruses and influenzas, commonly cause respiratory tract infections in humans. While in some humans, such viral infections are mild to moderate, in others the infections are severe and even fatal. Certain viruses, such as the novel coronavirus disease 2019 (COVID-19), have proven to be more fatal than other viral infections. As one example, the high level of contagiousness and high mortality rate for the coronavirus disease 2019 (COVID-19) have resulted in a pandemic that has already infected millions of people globally. The existence of rapid, accurate COVID-19 diagnostic tests could allow infected individuals to be quickly identified and isolated, which could assist with containment of the disease and allow appropriate public health measures to be enacted. In the absence of such diagnostic tests, COVID-19 may continue to spread unchecked throughout communities.

### SUMMARY

Provided herein are various embodiments of breakable swabs for sample collection. In some embodiments, the breakable swab is used with a number of diagnostic tests useful for detecting target nucleic acid sequences. The tests, as described herein, are able to be performed in a point-of care (POC) setting or home setting without specialized equipment. In some embodiments, the tests detect presence or absence of COVID-19 and/or an influenza virus and/or a target nucleic acid. In an embodiment, the influenza virus is an influenza A virus or an influenza B virus. In an embodiment, the target nucleic acid is a nucleic acid of a viral, bacterial, fungal, parasitic, or protozoan pathogen.

In some embodiments, a swab for sample collection includes an elongated body including a first end and a second end, a head portion extending from the first end to a breakable portion, and a stem portion extending from the second end to the breakable portion. The breakable portion is positioned along the elongated body between the head portion and the stem portion. The stem portion is configured to be detached from the head portion at the breakable portion.

According to some aspects, the head portion comprises a swab tip configured to hold a sample, and the stem portion comprises a handle configured to be handled by a user.

According to some aspects, the sample is a mucus sample or a saliva sample.

According to some aspects, the head portion is configured to be inserted into a nasal cavity or an oral cavity of a subject.

According to some aspects, a first cross-sectional width of the stem portion is greater than a second cross-sectional width of the breakable portion, the first cross-sectional width and the second cross-sectional width measured across a central axis extending along the elongated body.

According to some aspects, the stem portion is detached from the head portion after insertion of the head portion into a reaction tube.

According to some aspects, the head portion is inserted into a reaction tube, and the reaction tube including the head portion and the sample is heated above 37° C.

According to some aspects, the head portion is inserted into a reaction tube, and the reaction tube including the head portion and the sample is mechanically agitated.

According to some aspects, the swab is used with a rapid test that detects presence or absence of at least one pathogen selected from COVID-19, an influenza virus, or a target nucleic acid.

According to some aspects, the rapid test is an isothermal test including a lateral flow strip and result readout element.

According to some aspects, the breakable portion includes one or more features configured to lower a resilience of the breakable portion with respect to a resilience of the stem portion.

In some embodiments, a swab for sample collection includes a head portion extending from a first end of the swab to a breakable portion of the swab, and a stem portion extending from a second end of the swab to the breakable portion. The stem portion is configured to be detached from the head portion at the breakable portion, and a cross-sectional width of the breakable portion measured along a central axis of the swab is less than a cross-sectional width of the head portion.

According to some aspects, the cross-sectional width of the breakable portion is less than a cross-sectional width of the head portion.

In some embodiments, a method of processing a sample includes inserting a head portion of a swab into a nasal cavity or an oral cavity of a subject, inserting the head portion of the swab into a reaction tube, and separating the stem portion from the head portion. The head portion is positioned at one end of the swab. The swab includes a stem portion positioned at an opposing end of the swab from the head portion.

According to some aspects, a method of processing a sample includes mechanically agitating the reaction tube including the head portion of the swab.

According to some aspects, a method of processing a sample includes heating the reaction tube including the head portion of the swab above 37° C.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various nonlimiting embodiments when considered in conjunction with the accompanying figures.

### BRIEF DESCRIPTION OF THE DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic depicting an embodiment of a testing procedure described herein;

FIG. 2 is schematic depicting an embodiment of a sample collection swab;

FIG. 3 is a schematic depicting an alternative configuration of the sample collection swab of FIG. 2;

FIG. 4 is an inset of the schematic from FIG. 3;

FIG. 5 is a perspective view of an embodiment of a sample collection swab in a test cartridge;

FIG. 6 is a partial cross-sectional view of the cartridge of FIG. 5 taken along line 6-6; and

FIG. 7 shows, according to some embodiments, a flow chart for a method of performing a diagnostic test.

### DETAILED DESCRIPTION

Conventional nucleic acid tests for various diseases require trained medical professionals to collect samples and process those samples in a sterile environment in a laboratory. Such a process is time consuming, resulting in a delay in providing results to patients. Additionally, such tests require a patient to visit a location where a sample may be collected and transported in a sterile manner to an appropriate processing location. Travel to and from locations may risk spread of the disease being tested for and may inadvertently expose medical personnel to the disease.

While diagnostic tests for various diseases, including COVID-19, are known, such tests often require specialized knowledge of laboratory techniques and/or expensive laboratory equipment. For example, polymerase chain reaction (PCR) tests generally require skilled technicians and expensive, bulky thermocyclers. In addition, there is a need for diagnostic tests that are both rapid and highly accurate. Known diagnostic tests with high levels of accuracy often take hours, or even days, to return results, and more rapid tests generally have low levels of accuracy. Many rapid diagnostic tests detect antibodies, which generally can only reveal whether a person has previously had a disease, not whether the person has an active infection. In contrast, nucleic acid tests (i.e., tests that detect one or more target nucleic acid sequences) may indicate that a person has an active infection.

In view of the above, the inventors have recognized the benefits of a rapid diagnostic device that is usable by users who may not be medical professionals, inexpensive to operate (e.g., may not require costly and/or specialized equipment), and accurate. In particular, the inventors have recognized the benefits of breakable sample-collection swabs used with rapid diagnostic devices to allow a collected sample to be processed with a small footprint. The breakable swab may include a breakable portion located in between a swab tip (for sample collection) and a handle (for swab handling or manipulation), such that applying a force or other stressor to the swab may separate the swab into a head portion including the swab tip and a stem portion including the handle. The swab may be broken prior/during/after insertion into a reaction tube (e.g., a reservoir of a diagnostic device), such that the head portion may remain in the reaction tube while the stem portion may be removed to allow for a more compact diagnostic device. Separation of the head portion from the stem portion may also reduce the risk of contamination from handling. Furthermore, the elongated form factor of the handle may also allow a user to accidentally knock the swab out of the reaction tube, which may compromise the integrity of the diagnostic test. The breakable swab may be used with diagnostic devices which allow users to perform tests and receive results in a rapid manner without necessarily requiring input from trained medical staff. Telemedicine, or applications may be employed to further enhance the usability of the rapid diagnostic test, such that a variety of diseases such as COVID-19, influenza, (or any target nucleic acid) may be tested for in an at-home or point-of-care environment.

The present disclosure provides breakable sample collection swabs for use with diagnostic devices, systems, and methods for rapidly detecting one or more target nucleic acid sequences (e.g., a nucleic acid sequence of a pathogen, such as SARS-CoV-2, an influenza virus, fungal pathogens, parasitic pathogens, protozoan pathogens, etc.). A diagnostic system, as described herein, may be self-administrable. Of course, diagnostic devices described herein may also be administered by a technician, a medical professional, or any other suitable professional in a point-of-care environment. A diagnostic device used with the breakable sample collection swab may comprise a plurality of fluid reservoirs including one or more solutions (e.g., lysis reagents, nucleic acid amplification reagents, CRISPR/Cas detection reagents, decontamination reagents, UDG, LAMP, RPA, NEAR), readout elements, or any other suitable components which enable a rapid diagnostic test. Results of the readout elements may be self-readable, or automatically read by a computer algorithm. In certain embodiments, the diagnostic device further. The diagnostic device may also comprise an integrated heater, or the diagnostic system may comprise a separate heater to employ fast and accurate results associated with isothermal amplification techniques.

Described herein are some embodiments of a breakable swab for sample collection. The breakable swab may include a breakable portion located in between a head portion and a stem portion. The head portion may span from one end of the swab (which may include a swab tip for sample collection) to the breakable portion. The stem portion may span from an opposing end of the swab (which may include a handle for swab manipulation and handling) to the breakable portion. The breakable portion may allow a user to separate or detach the head portion and the stem portion.

In some embodiments, a breakable swab may be used for collection of a mucus or saliva sample by inserting into a nasal cavity or oral cavity of a person or subject. The swab may be used in combination with a rapid diagnostic test to detect presence or absence of COVID-19 and/or an influenza virus and/or a target nucleic acid. The swab, which may include collected sample at one end (e.g., on a swab tip) may then be inserted into a reaction tube of a diagnostic device (or any other suitable sample-receiving system) for conducting a diagnostic test. In some embodiments, the diagnostic test may be an isothermal test including a lateral flow strip and result readout element.

The head portion of the swab may be inserted into the reaction tube and may remain in the reaction tube for further processing upon separation or detachment from the stem portion. In some embodiments, the reaction tube containing the head portion may be heated to a desired temperature. In some embodiments, the reaction tube containing the head portion may be mechanically agitated to release viral (or bacterial or fungal or other pathogenic or nucleic acid of interest) particles, enabling capture of genetic material and/or lysis of pathogenic material. In some embodiments, the reaction tube may be both heated and mechanically agitated. Of course, other means of releasing sample particles from the swab tip are also contemplated, as the present disclosure is not so limited.

The breakable swab and the diagnostic device may be self-administrable. Software, which may be downloaded to a device, may be provided to guide a user through administration of the testing. Test results may be uploaded, manually or automatically, to a device or communicated remotely through a network. Of course, the swab and/or device may also be operated by a secondary user (e.g., a healthcare professional). In some embodiments, the diagnostic device may be capable of detecting presence of COVID-19 and/or influenza and/or a target nucleic acid within 60 minutes. In some embodiments, the diagnostic device may provide a visual and/or audio illustration representing positive or negative presence of COVID-19, and/or influenza and/or a target nucleic acid.

In some embodiments, the diagnostic test may not require special laboratory equipment—that is, the test may be performed in a non-laboratory setting (e.g., a home). In some embodiments, the diagnostic device capable of performing a diagnostic test may be self-contained. For example, the device may comprise a cartridge and/or a series of lateral flow strips that are sealed. Exemplary embodiments of a diagnostic device are provided in further detail below. In some embodiments, the diagnostic test may be an over-the-counter (OTC) test for consumer use as an aid in the diagnosis of an infection (e.g., SARS-CoV-2 infection in individuals with signs and symptoms of infection). The results from a test may be useful for the identification of infectious material (e.g., SARS-CoV-2 RNA). For example, the SARS-CoV-2 RNA is generally detectable in samples from human subjects (e.g., anterior nares specimens) during the acute phase of infection. Positive results are indicative of active infection.

A diagnostic test may be envisioned to comprise the steps of collecting a sample, processing the sample, and analyzing the sample. Sample processing may occur in any suitable manner or order. In some embodiments, sample processing includes lysing the sample and amplifying the nucleic acids. Analysis of the sample, e.g., determination of whether the sample is positive or negative for one or more pathogens, may comprise the use of a readout element (e.g., a lateral flow assay strip, as known in the art). The readout element may be configured to identify one or more pathogens, such as COVID-19, influenza type A, influenza type B, or any other pathogen. In some embodiments, the diagnostic test may be guided by a companion mobile application (“app”), for example, on a cellular phone (e.g., smartphone).

In some embodiments, the diagnostic tests used with the breakable swabs described herein have one or more of the following attributes:
a. Detects with saliva or nasal swab or any other sample from a subject
b. Utilizes lyophilized reagents
c. Includes visual readout (legible with naked eye or by smartphone)
d. Utilizes positive controls
e. Can be performed at home with no auxiliary equipment
f. At least 1 picomolar sensitivity (e.g., 10 aM sensitivity)
g. Can be performed at room temperature or at human body temperature (e.g., 37° C.)
h. Includes test strip readout or a colorimetric readout

The diagnostic tests described in combination with the breakable swab of the current disclosure may be used outside the hospital or medical environment (e.g., at an individual's home). Therefore, the tests described herein require minimal outside interference and involvement (e.g., a home-testing device).

Of course, the utility of the breakable swab described herein is not limited to rapid diagnostic tests or any other classification of diagnostic tests. Furthermore, the breakable swab described herein may be used in non-diagnostic applications. The breakable swab described herein may be used in any suitable application (including applications which do not explicitly require breakage of the breakable portion of the swab), as the present disclosure is not so limited.

### Sample Collection

In some embodiments, a sample to be tested comprises a sample from a subject (e.g., a human subject, an animal subject). Exemplary samples include bodily fluids (e.g., mucus, saliva, blood, serum, plasma, amniotic fluid, sputum, urine, fecal, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, the sample comprises a nasal secretion.

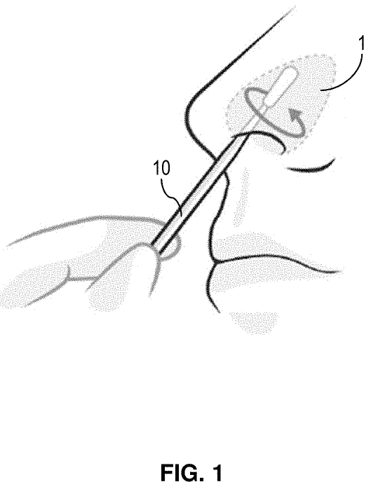

As shown in FIG. 1, in certain instances, the sample may be an anterior nares 1 specimen. An anterior nares specimen may be collected from a subject by inserting a portion of a sample collection swab 10 into one or both nostrils of the subject (e.g., a human subject) for a period of time (e.g., approximately 10 seconds). The swab 10 may be swirled or otherwise brought into contact with various surfaces of the nostril to collect a sufficient volume of sample.

In other embodiments, the sample comprises a cell scraping. In certain embodiments, the cell scraping may be collected from the mouth or interior cheek. The cell scraping may be collected using a brush or scraping device formulated for this purpose, and positioned at one end of the swab. In some embodiments, a sample may be self-collected by the subject or may be collected by another individual (e.g., a family member, a friend, a coworker, a health care professional) using a sample collection swab described herein.

### Breakable Swab

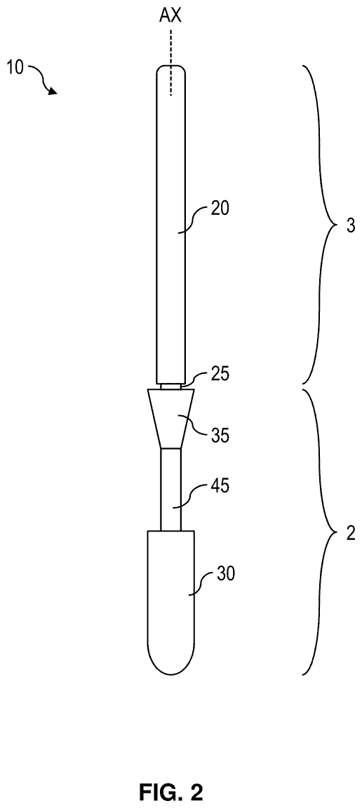

According to some embodiments, a breakable sample collection swab (hereinafter referred to as “swab”) may include a generally elongated body extending from one end to an opposing end along a central axis AX. The swab 10 may include a head portion 2 and a stem portion 3, as shown in FIG. 2. The head portion 2 may span from a first end of the swab 10, which may include a swab tip 30 for sample collection, to a breakable portion 25. The swab tip 30 may be used for sample collection (e.g., by inserting into desired sample site), sample transport (e.g., by holding sample while swab 10 is manipulated), and for sample deposition (e.g., by inserting swab tip 30 into a reaction tube). The stem portion 3 may span from the opposing end of the swab 10, which may include a handle 20, to a breakable portion 25. The head portion may include the swab tip 30, connecting portion 45, flange 35, and a portion of the breakable portion 25. The stem portion 3 may include the handle 20 and a portion of the breakable portion 25. The handle 20 may be used to manipulate the swab 10 (e.g., place into nostril, place into testing cartridge) without contacting the swab tip 30, which may contaminate any sample present on the swab tip 30. In some embodiments, the handle 20 may extend along the central axis AX. In some embodiments, the head portion may include a swab tip directly connected to a flange. In other embodiments, the head portion may include a swab tip directly connected to the breakable portion. In other embodiments still, the head portion may include a swab tip directly connected to the breakable portion. In some embodiments, the breakable portion may be located in between a handle and a swab tip. It should be appreciated that the current disclosure is not limited by the structure or arrangement of structures of the head portion.

It should be appreciated that the head portion 2 (not including the swab tip 30) and the stem portion 3 may be a continuous body prior to separation. In other words, the swab may be formed as a unitary elongated rod, which may include geometric variations along a central axis. In other embodiments, the head portion 2 and the stem portion 3 may be assembled prior to use (e.g., at a manufacturing facility). Of course, other suitable assembly arrangements are also contemplated, as the present disclosure is not so limited.

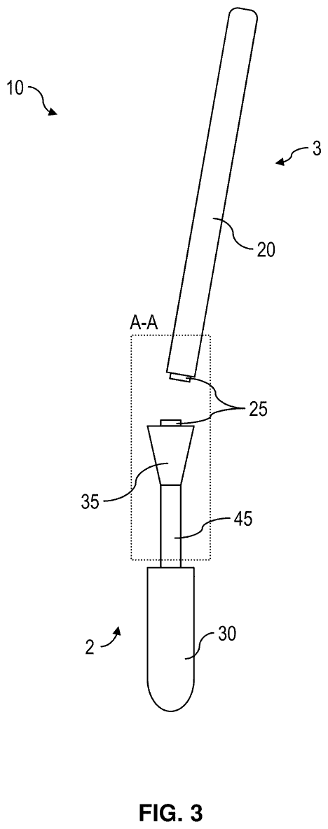

In some embodiments, the swab 10 may be breakable at the breakable portion 25. In other words, the swab 10 may be separated into the head portion 2 and stem portion 3 upon breakage of the breakable portion 25, as shown in FIG. 3. The breakable portion 25 may facilitate separation of a head portion 2 and a stem portion 3 of a swab 10 upon application of a relatively low amount of force (e.g., a manually applied force). In some embodiments, the threshold breakage force may be equivalent to the force required to break a popsicle stick. The threshold breakage force may be of any suitable magnitude which prevents breakage prior to use and allows breakage during intended breakage. In other words, the breakable portion 25 may be sufficiently robust to reduce the likelihood of breakage prior to use (e.g., during storage, distribution, sample collection). FIG. 3 shows an exemplary swab 10 after application of such a force, wherein stem portion 3 and head portion 2 have been separated.

In some embodiments, the threshold breakage force of the breakable portion may be lower than a threshold breakage force of any other portion of the swab. The threshold breakage force of the breakable portion may also be lower than a threshold force which may warp or otherwise undesirably deform any other portion of the swab (e.g., the handle). In some embodiments, the threshold breakage force of the head portion and/or the stem portion may be at least 10%, 20%, 25%, 30%, 33%, 40%, 50%, 60%, 67%, 70%, 75%, 80%, 90%, 100%, 125%, 150%, 175%, 200%, 300%, 400%, 500%, 750%, 1000%, or any other percentage greater than the threshold breakage force of the breakable portion. Embodiments in which the threshold breakage force of the head portion and/or the stem portion may less than or equal to 1000%, 750%, 500%, 400%, 300%, 200%, 175%, 150%, 125%, 100%, 90%, 80%, 75%, 70%, 67%, 60%, 50%, 40%, 33%, 30%, 25%, 20%, 10%, or any other percentage greater than the threshold breakage force of the breakable portion. Combinations of the foregoing ranges are also contemplated. In some embodiments, the threshold breakage force of the head portion and/or the stem portion may be between 10% and 1000%, 100% and 500%, 20% and 80%, 50% and 200%, or any other suitable range of percentages greater than the threshold breakage force of the breakable portion.

In some embodiments, the breakable portion 25 may include any suitable structure to encourage the swab 10 to break at the breakable portion 25, which may facilitate separation of the stem portion 3 and head portion 2. For example, the breakable portion 25 may be a localized stress maximum, which may lead to the breakage of the breakable portion 25 prior to other components of the swab 10 (e.g., stem portion 3). In other words, the breakable portion 25 may break at lower forces than the stem portion 3 or any other portion of the swab 10. In this way, the stem portion 3 and/or head portion 2 may be structurally sound individual components even after separation. In some embodiments, applying a bending force to the swab 10 may cause the breakable portion 25 to break, whereas in other embodiments, applying a torsional (e.g., twisting) force to the swab 10 may cause the breakable portion 25 to break. In some embodiments, a bending moment may be applied to the swab 10 when the head portion 2 is located in a reaction tube. Accordingly, the handle 20 may lead the head portion 2 to a sidewall of a reaction tube or other suitable housing. The sidewall may prevent the head portion 2 from bending along with the handle 20, and may therefore result in breakage of the breakable portion 25. Of course, combinations of bending, torsional, compressive, or any other suitable applied forces are also contemplated to break the breakable portion 25.

In some embodiments, the head portion 2 and the stem portion 3 may be separated or detached without the user having to handle the head portion 2. In this way, risk of contamination with the sample during handling may be reduced.

In some embodiments, the breakable portion 25 may be formed of a different material from the other portions of the swab 10. In this way, the breakable portion 25 may be more susceptible to breakage than the handle 20 or flange 35 (or any other portion of the swab 10) due to material properties. For example, the breakable portion 25 may be formed of a more brittle and/or less elastic material than other portions of the swab 10. In this way, the breakable portion 25 may break (thereby separating the head portion 2 from the stem portion 3) at lower forces than the remaining portions of the swab 10. In some embodiments, the breakable portion 25 may break due to external stresses such as heat or chemical stresses such as pH, hydration, or any other stresses. It should be appreciated that the breakable portion 25 may be configured to allow for controllable breakage, such that the intact swab 10 may be used for sample collection and a separated swab 10 (as shown in FIG. 3, for example) may be used during a subsequent diagnostic test.

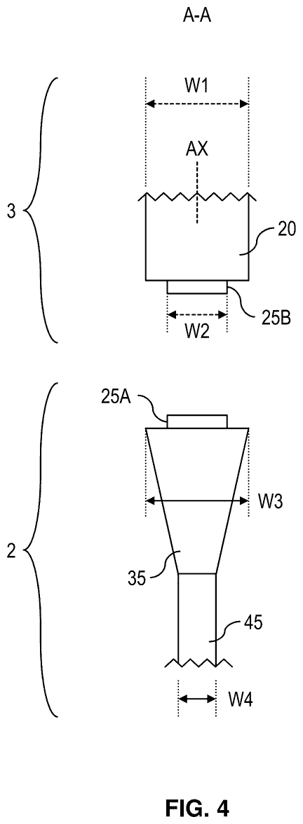

Of course, embodiments in which the breakable portion 25 may be more susceptible to breakage due to a combination of structural and material differences are also contemplated. Accordingly, breakable portion 25 may comprise any feature or combination of features to facilitate separation of stem portion 3 and head portion 2. In some embodiments, a breakable portion may include one or more cutouts, scored lines, nicks, variations in cross-sectional area, variations in cross-sectional shape, perforations, or any other feature or combination of features. These features may enhance stress concentrations at the breakable portion 25 and/or render the breakable portion 25 weaker than other portions of the swab 10. In some embodiments, the breakable portion 25 may split into two or more portions 25A, 25B upon breakage (shown in FIG. 4), such that a portion 25A of the breakable portion 25 may be attached to the head portion 2 and another portion 25B of the breakable portion 25 may be attached to the stem portion 3 after separation. In some embodiments, the breakable portion 25 may separate from the head portion 2 and/or stem portion 3, such that no remnant of the breakable portion 25 may remain on the swab 10 after breakage.

In some embodiments, the breakable portion may be narrower than nearby portions of the swab, taken along a central axis of the swab. In some embodiments, the narrowing of the breakable portion (or any other portion) of the swab may be quantified by a narrowing dimension of the portion in question, taken along a central axis. For example, the dimension is a diameter of the portion, which is smaller for the breakable portion than a nearby portion. In some embodiments, the narrowing dimension may be a cross-sectional width taken as a dimension (e.g., the largest dimension) of the cross-section of the portion taken along a central axis. The cross-sectional width may be measured across the central axis. In embodiments where the breakable portion or any other portion are substantially cylindrical, the cross-sectional width may correspond to the diameter of the portion in question. In embodiments where the portion is non-cylindrical (e.g., non-circular cross-section), the cross-sectional width may be a dimension of the cross-sectional shape taken along the central axis. It should be appreciated that the narrowing of the breakable portion (or any other suitable portion) may be quantified using any suitable dimension, as the present disclosure is not so limited.

As shown in FIG. 4, an inset of FIG. 3 within boundary A-A, the handle 20 may include a cross-sectional width (hereinafter referred to as “width”) W1, the breakable portion 25 may include a width W2, the flange portion 35 may include a maximum width W3, and the connecting portion 45 may include a width W4. The widths W1, W2, W3, and W4 are taken along the central axis AX, as shown in FIG. 4. In some embodiments, width W1 of the handle 20 may be sized to be comfortably manipulated by a user (e.g., may be sized appropriate to be held by one hand), and/or may be sized to interact with a diagnostic device. In some embodiments, width W2 of the breakable portion 25 may be less than width W1 of the handle 20. Accordingly, the breakable portion 25 may be more susceptible to breakage compared to handle 20. In some embodiments, width W2 of the breakable portion 25 may be less than width W3 of the flange portion W3. In some embodiments, flange portion 35 may be widest at its interface with the breakable portion 25. In this way, the drastic change in width between the flange portion 35 and breakable portion 25 (e.g., between width W3 and width W2) may encourage the swab 10 to break at the breakable portion 25. In some embodiments, width W2 of the breakable portion 25 may be less than the width W4 of the connecting portion W4. Accordingly, the breakable portion 25 may be more susceptible to breakage and/or may be less resilient than connecting portion W4. The relationship between widths W1, W3, and W4 may be less significant to the breakage of the breakable portion 25. Accordingly, the handle 20, connecting portion 45, and the flange 35 may have any suitable width taken along central axis AX, as the present disclosure is not so limited.

In some embodiments, one or more structural features (including cross-sectional size, area, or cutouts, etc. as described previously), material variations, and/or any combination of the two may lower the resilience of the breakable portion 25 with respect to the stem portion 3. In some embodiments, one or more structural features and/or material variations may lower the resilience of the breakable portion 25 with respect to the head portion 2. As described previously, the breakable portion 25 may be more susceptible to breakage and/or may be less resilient than other portions of the swab 10 (e.g., handle 20, flange 35, connecting portion 45) due to any suitable combination of structure (including size and/or features to reduce resilience of the breakable portion 25) and material.

Of course, embodiments in which various portions of the swab 10 include variable widths are also contemplated. For example, a handle may be tapered or curved to allow ergonomic handling of the swab. Any portion of the swab may be tapered linearly or non-linearly for ergonomic, esthetic, or functional (e.g., to improve engagement with sample collection site and/or diagnostic device). In some embodiments, changes in cross-sectional area (including cross-sectional width and/or shape) along the central axis AX may be discrete (as shown in FIGS. 2-4) or may be gradual, such that each portion may smoothly transition into the next, along the central axis. In some embodiments, smooth and/or gradual changes in cross-sectional area and/or shape of various portions of the swab may allow for ergonomic handling and may facilitate manufacturing of the swab. Of course, the swab described herein is not limited by the variation of cross-sectional area and/or shape, as any suitable swab structure may be used. Accordingly, the comparison of sizing (e.g., cross-sectional width) of the swab components described above is by way of example only. The breakable portion 25 may include any structure (including size and/or feature) or material to render it more susceptible to breakage than other components of the swab.

It should be appreciated that swab tip 30 may have any suitable width (not shown) irrespective of the remaining components of the swab 10. The swab tip 30 may be sized to accommodate the sampling region which it may be inserted into (e.g., a nasal cavity) and/or a diagnostic test which it may also interact with. For example, the swab tip 30 may be sized to fit within a reaction tube of a compact diagnostic device. Accordingly, the present disclosure is not limited by the size of the swab tip 30. It should also be appreciated that the present disclosure is not limited by the height of any of the components of the swab 10 (e.g., handle 20, tip 30, etc.) taken along the central axis AX. In other words, any component of the swab 10 may be any suitable height taken along the central axis AX.

In certain embodiments, a swab tip 30 may include a material and/or structure in order to collect the sample from a subject. In some embodiments, the swab tip 30 may be an absorbent material. The absorbent material may be any absorbent material suitable for oral or nasal use. Non-limiting examples of suitable absorbent materials include cotton, filter paper, cellulose-based materials, polyurethane, polyester, rayon, nylon, microfiber, viscose, and alginate. In some embodiments, the swab tip 30 may be formed of a foam material and/or may be formed of flocked fibers. Of course, the material composition of swab tip 30 may be selected based on the sample properties (e.g., the swab tip 30 material may be compatible with the sample to reduce risk of contamination), sample source properties (e.g., the swab tip 30 may be formed of a material to allow the swab to explore cavernous regions of a nasal cavity), reagent properties (e.g., the swab tip 30 may not react with the reagent), or any other property. The swab tip 30 may be formed of any suitable material which is capable of collecting a sample and transferring said sample to a diagnostic device.

The handle 20, connecting portion 45, flange 35, breakable portion 25, or any combination of the components of the swab 10 may be formed from any suitable material. In some embodiments, one or more components of the swab 10 comprises a thermoplastic material (e.g., a polystyrene, a polyethylene, a polypropylene, a polystyrene, an olefin), a metal (e.g., aluminum), wood, paper, or another type of material. In some embodiments, one or more components of the swab 10 comprises one or more markings. The markings may, in some instances, indicate the appropriate depth of insertion (e.g., into a nasal cavity) during sample collection. In some embodiments, the swab 10 comprises means for processing and analyzing the sample (e.g., amplification and detection, as described in greater detail below) in any component or combination of components of the swab 10. In some embodiments, at least a portion of the swab 10 may be wrapped in a material (e.g., plastic) to ensure sterility until use. In certain embodiments, the swab tip 30 may be pre-moistened.

In some embodiments, swab 10 includes contact between the swab tip 30 and a desired sample. The swab 10 may then be placed into a reaction tube of a diagnostic device (or any other suitable reaction tube, e.g., an Eppendorf tube), bringing the sample into fluid communication with any reagents (e.g., lysis reagent) which may reside in the reaction tube. The stem portion 3 may then be separated from the head portion 2 (while the head portion 2, including swab tip 30) remains in the reaction tube for further processing. The stem portion 3 may be separated by a user applying a bending force to the swab 10, a twisting force to the swab 10, any combination of applied stresses, or any other stresses which may induce breakage of the swab 10 at the breakable portion 25. The stem portion 3 may be separated from the head portion 2 without the user having to contact the head portion 2, which may reduce contamination risk. As described previously, the breakable portion 25 may separate the head portion 2 and stem portion 3 in an autonomous manner upon exposure to fluid and/or any other condition.

By keeping the head portion 2 and collected sample within a reaction tube or other component of a diagnostic or sample-receiving device, the swab tip 30 may be subjected to further processing without contamination of the sample. For example, in one embodiment, the sample-containing head portion 2 may be placed within a reaction tube, the stem portion 3 of the swab 10 may be broken and removed, and the reaction tube may be covered by a lid or cap. The head portion 2 and the sample, uncompromised, may then be subject to further processing. In some embodiments, the reaction tube may comprise one or more reagents for further processing. In some embodiments, the reaction tube comprises an assay. In certain embodiments, the reaction tube (e.g., comprising the head portion 2 and the sample) may be heated to a desired temperature (e.g., by a heat source). In certain embodiments, the reaction tube may be mechanically agitated. The mechanical agitation may be performed manually or using laboratory equipment (e.g., a vortex mixer). In some cases, mechanical agitation of the reaction tube advantageously promotes release of particles of interest (e.g., viral, bacterial, fungal, or other pathogenic particles, or a nucleic acid of interest) from the swab tip 30. In some cases, such release of particles may facilitate capture of genetic material and/or lysis of pathogenic cells.

Several non-limiting examples of tests with which the breakable sample collection swab may be used are provided below. It should be appreciated that the breakable sample collection swab described herein may be used with any suitable diagnostic device or other sample-receiving system.

### Sample

In some embodiments, the sample collected on the swab tip may be a saliva sample. The saliva sample may have any suitable volume. In some embodiments, the volume of the sample may be at least 1 mL, at least 1.5 mL, at least 2 mL, at least 2.5 mL, at least 3 mL, at least 3.5 mL, at least 4 mL, or any other suitable volume. In some embodiments, the sample has a volume in a range from 1 mL to 3 mL, 1 mL to 4 mL, or 2 mL to 4 mL, or any other suitable range. In some embodiments, the saliva sample has a volume of 1 mL, 1.5 mL, 2 mL, 2.5 mL, 3 mL, 3.5 mL, 4 mL, or any other suitable volume. Saliva has been found to have a mean concentration of SARS-Cov-2 RNA of 5 fM (Kai-Wang To et al., 2020), an amount which may be detectable by diagnostic devices using the breakable swabs described herein.

In some embodiments, the concentration of pathogen RNA (e.g., COVID-19 RNA) in the sample (e.g., a saliva sample, a mucus sample) may be at least 5 aM, at least 10 aM, at least 15 aM, at least 20 aM, at least 25 aM, at least 30 aM, at least 35 aM, at least 40 aM, at least 50 aM, at least 75 aM, at least 100 aM, at least 150 aM, at least 200 aM, at least 300 aM, at least 400 aM, at least 500 aM, at least 600 aM, at least 700 aM, at least 800 aM, at least 900 aM, at least 1 fM, at least 5 fM, at least 10 fM, at least 15 fM, at least 20 fM, at least 25 fM, at least 30 fM, at least 35 fM, at least 40 fM, at least 50 fM, at least 75 fM, at least 100 fM, at least 150 fM, at least 200 fM, at least 300 fM, at least 400 fM, at least 500 fM, at least 600 fM, at least 700 fM, at least 800 fM, at least 900 fM, at least 1 pM, at least 5 pM, or at least 10 pM. In some embodiments, the concentration of pathogen RNA (e.g., COVID-19 RNA) may be 10 pM or less, 5 pM or less, 1 pM or less, 500 fM or less, 100 fM or less, 50 fM or less, 10 fM or less, 1 fM or less, 500 aM or less, 100 aM or less, 50 aM or less 10 aM or less, or 5 aM or less. In some embodiments, the concentration of pathogen RNA (e.g., COVID-19 RNA) in the sample may be in a range from 5 aM to 50 aM, 5 aM to 100 aM, 5 aM to 500 aM, 5 aM to 1 fM, 5 aM to 10 fM, 5 aM to 50 fM, 5 aM to 100 fM, 5 aM to 500 fM, 5 aM to 1 pM, 5 aM to 10 pM, 10 aM to 50 aM, 10 aM to 100 aM, 10 aM to 500 aM, 10 aM to 1 fM, 10 aM to 10 fM, 10 aM to 50 fM, 10 aM to 100 fM, 10 aM to 500 fM, 10 aM to 1 pM, 10 aM to 10 pM, 100 aM to 500 aM, 100 aM to 1 fM, 100 aM to 10 fM, 100 aM to 50 fM, 100 aM to 100 fM, 100 aM to 500 fM, 100 aM to 1 pM, 100 aM to 10 pM, 1 fM to 10 fM, 1 fM to 50 fM, 1 fM to 100 fM, 1 fM to 500 fM, 1 fM to 1 pM, 1 fM to 10 pM, 5 fM to 10 fM, 5 fM to 50 fM, 5 fM to 100 fM, 5 fM to 500 fM, 5 fM to 1 pM, 5 fM to 10 pM, 10 fM to 100 fM, 10 fM to 500 fM, 10 fM to 1 pM, 10 fM to 10 pM, 100 fM to 500 fM, 100 fM to 1 pM, 100 fM to 10 pM, or 1 pM to 10 pM.

The sample, in some embodiments, may be from a subject who is suspected of having the disease(s) the test screens for, such as a coronavirus (e.g., COVID-19) or influenza (e.g., influenza type A or influenza type B). Other indications, as described herein, are also envisioned. In some embodiments, the subject may be a human. Subjects may be asymptomatic, or may present with one or more symptoms of the disease(s). Symptoms of coronaviruses (e.g., COVID-19) include, but are not limited to, fever, cough (e.g., dry cough), generalized fatigue, sore throat, runny nose, nasal congestion, muscle aches, and difficulty breathing (shortness of breath). Symptoms of influenza include, but are not limited to, fever, chills, muscle aches, cough, congestion, runny nose, headaches, and generalized fatigue. In some embodiments, the subject may be asymptomatic. In some embodiments, the subject has had contact within the past 14 days with a person that has tested positive for the virus.

In some embodiments, the head portion containing the sample may be placed in a reaction tube comprising a rehydration buffer. The rehydration buffer, in some embodiments, comprises Tris pH 8.0, poly(ethylene glycol), magnesium acetate tetrahydrate, potassium acetate, and nuclease free water. As an example, the rehydration buffer may comprise: 25 mM Tris buffer, 5% (w/v) poly(ethylene glycol) 35,000 kDa, 14 mM magnesium acetate tetrahydrate, 100 mM potassium acetate, and >85% volume nuclease free water. In some embodiments, a buffer (e.g., phosphate-buffered saline (PBS)) may be added to the reaction tube.

### Sample Processing

As described previously, a swab may be used in combination with a diagnostic device (for performing a diagnostic test) which may include one or more reagents with may amplify, react, and/or process sample container on the swab (e.g., on the swab tip). In some embodiments, one or more of the reagents may be configured to lyse the sample.

Lysis may be performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. In some embodiments, the one or more lysis reagents comprise one or more detergents. In some cases, one or more lysis reagents may be lyophilized. In certain embodiments, lysis may be accomplished by contacting the sample with a lyophilized lysis pellet (also referred to as a lysis bead). The lyophilized lysis pellet may contain one or more lyophilized lysis reagents and may be added to any of the tests provided herein. As an example, a lyophilized lysis pellet may be held in a cap/lid designed to release the pellet into solution after the sample has been mixed with a buffer (e.g., a rehydration buffer).

In some embodiments, cell lysis may be accomplished by applying heat to a sample (thermal lysis). In certain instances, thermal lysis may be performed by applying a lysis heating protocol comprising heating the sample at one or more temperatures for one or more time periods using any heater described herein. In some embodiments, a lysis heating protocol comprises heating the sample at a first temperature for a first time period.

In some embodiments, cell lysis may be accomplished by applying heat to a sample (thermal lysis). In some instances, a sample (e.g., a sample within a reaction tube) may be exposed to a heat source. In some cases, the sample may be heated to a temperature of at least 30° C., at least 37° C., at least 40° C., at least 55° C., at least 60° C., at least 65° C., at least 70° C., at least 75° C., at least 90° C., or higher. In some embodiments, the sample may be heated to a temperature of 30° C., 37° C., 40° C., 55° C., 60° C., 61° C., 62° C., 63° C., 64° C., 65° C., 66° C., 67° C., 68° C., 69° C., 70° C., 75° C., 90° C., or higher. In certain cases, the sample may be heated to a temperature in a range from 30° C. to 55° C., 30° C. to 75° C., 30° C. to 90° C., 37° C. to 55° C., 37° C. to 75° C., 37° C. to 90° C., 55° C. to 75° C., 55° C. to 90° C., 60° C. to 75° C., 60° C. to 90° C., or 75° C. to 90° C. The heat source may be any device capable of heating the sample. In certain embodiments, the heat source may be a USB-powered heat source, a hot plate, a heating coil, or a hot water bath. In some embodiments, the heat source may be an off-the-shelf consumer-grade device. In some embodiments, the heat source may be a thermocycler or other specialized laboratory equipment known in the art. The heating source may, in some cases, be contained within a thermally-insulated housing to ensure user safety. In some embodiments, the heat source comprises at least two temperature zones. In some embodiments, the heat source may be an off-the-shelf consumer-grade heating coil connected to a microcontroller that may be used to switch between the two temperature zones. In some embodiments, the two temperature zones are 60° C.-90° C. and 30-40° C. In certain instances, the first temperature zone has a temperature of 65° C., and the second temperature zone has a temperature of 37° C.

Lysis of cells within the sample may be performed by any suitable lysis method known in the art. Lysis may be performed on paper (Rodriguez et al., Anal Chem. 2015 Aug. 4; 87(15): 7872-7879) or in a test tube (Ma et al., Mol Cell Probes 2018 October; 41:27-31). In some embodiments, cell lysis may be performed by adding one or more lysis reagents (e.g., one or more enzymes and/or detergents) to a reaction tube. Various lysis reagents are known in the art. A number of such lysis reagents are commercially available (e.g., from Sigma-Aldrich, ThermoFisher Scientific, etc.). In some embodiments, cell lysis may be performed at room temperature (e.g., 20° C.-22° C.). In other embodiments, cell lysis may be performed at higher temperatures, such as the boiling point temperature of a detergent used for lysing (e.g., 90° C.).

### Diagnostic Devices

According to some embodiments, diagnostic systems comprise a sample-collecting component (e.g., a swab according to some embodiments of the current disclosure) and a diagnostic device. In some cases, the diagnostic device comprises a detection component (e.g., a lateral flow assay strip). In certain embodiments, the diagnostic device further comprises one or more reagents (e.g., lysis reagents, nucleic acid amplification reagents, CRISPR/Cas detection reagents). Each of the one or more reagents may be in liquid form (e.g., in solution) or in solid form (e.g., lyophilized, dried, crystallized, air jetted). The diagnostic device may also comprise an integrated heater, or the diagnostic system may comprise a separate heater configured to heat one or more fluid containers or any other component of a diagnostic system. In some embodiments, a heater may be a printed circuit board (PCB) heater that may be integrated into a diagnostic device.

In some embodiments, one or more of processing, detecting, or analyzing steps of a diagnostic test are performed with a cartridge. In some embodiments, a sample may be processed using the various reservoirs or chambers of the cartridge, each of which are interconnected via channels molded into cartridge plastic. In some embodiments, a silicone peristaltic layer forms “pump lanes” associated with various channel connections, which by action of pumping with a user-operated roller pumping tool, drives sample and reagent between reservoirs at the appropriate times. Passive valves in each pump lane isolate the reservoirs during non-pumping events. Heat (e.g., via a PCB heater) may be applied to the underside during lysis and amplification. Critically, the amplification reservoir may be sealed from atmosphere and must have some of its air evacuated to allow ingress of pumped lysate. Additionally, a recirculation-type pumping operation allows amplified sample to come into contact with the read-out strip without exposing any of these components to atmosphere.

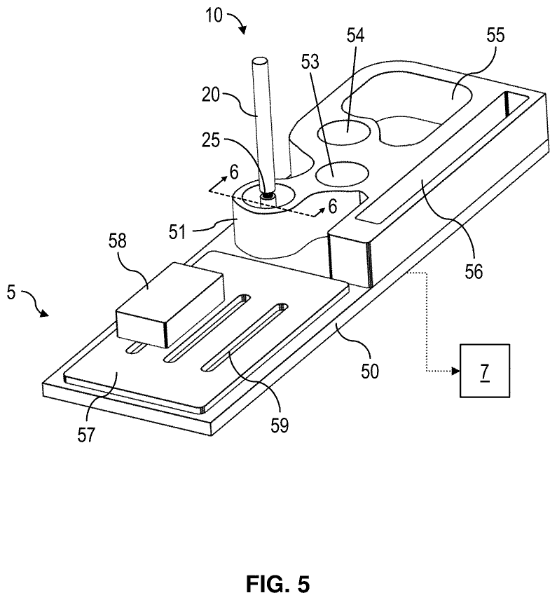

FIG. 5 shows a swab 10 installed in a cartridge 5 according to some embodiments. The cartridge 5 includes a reaction tube 51, a dilution buffer reservoir 53, an amplification reservoir 54, an air expansion reservoir 55, a readout element 56, a seal plate 57 with associated pump channel 59. In some embodiments, the reaction tube 51 may be a lysis reservoir. Any of the reservoirs of the cartridge 5 may be pre-filled with a liquid or lyophilized reagent. Of course, other arrangements of the various reservoirs and structures of the cartridge 5 are also contemplated, as the present disclosure is not so limited.

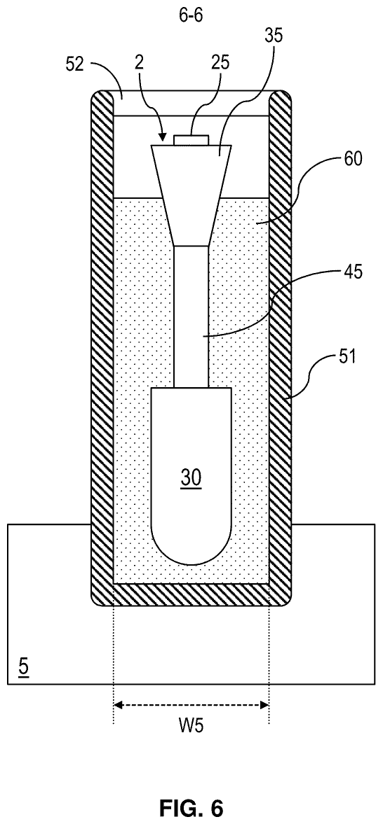

In some embodiments a swab 10 may be inserted into the reaction tube 51 by its head portion (not shown). The reaction tube 51 (and any other reservoirs) may include a cap (see cap 52 in FIG. 6) or a membrane to protect any fluid which may be located within the tube. In some embodiments, the cap may be a puncturable or frangible film, such that insertion of the swab 10 into the tube 51 may puncture the film. In other embodiments, the cap may be removeable such that a user may remove the cap prior to inserting the swab 10 into the tube 51. The cap may be used to seal the tube 51 after the stem portion of the swab has been detached from the head portion, as will be described in greater detail below.

In operation, a sample (e.g., anterior nares specimen) may be collected from a subject using the head portion 2 (including a swab tip 30) of a swab 10. An exemplary depiction of sample collection is shown in FIG. 1. The swab 10 may then be inserted into the reaction tube 51 and swirled around (or otherwise manually agitated in the tube 51 using handle 20) to deposit sample from the head portion into the lysis solution of the reaction tube 51. In some embodiments, the reaction tube 51 may subsequently be heated to lyse the sample (in other embodiments, the lysis reservoir comprises enzymes and/or detergents that lyse the sample at room temperature). The lysate may then be transported to the amplification reservoir 54. As shown in FIG. 5, the cartridge 5 may include a pumping tool 58 to transport fluid between the various regions of the cartridge 5. In some embodiments, the pumping tool 58 may be a single-finger operated pumped assembly. A user may slide the pumping tool 58 along the appropriate pump channel 59 in sequential order according to pre-set instructions (which may be displayed on a mobile application in some embodiments).

In some embodiments, the swab 10 may be separated into a head portion 2 and stem portion 3 (see FIG. 2) by breaking breakable portion 25 at any point during the diagnostic test. For example, the swab 10 may be broken prior to the heating process. Breaking the swab 10 allows the reaction tube 51 to be sealed (e.g., by a cap which may cover the tube and head portion 2) to limit evaporation from the tube 51 during heating. In other embodiments, the reaction tube 51 may include a self-healing seal such that insertion of a swab 10 (including the handle 20) may also limit evaporation from the tube 51 during heating.

FIG. 6 shows a cross-sectional view of an exemplary swab head portion 2 inserted into a reaction tube 51 after being separated or detached from a stem portion through a breakable portion 25. In some embodiments, the reaction tube 51 may include one or more reagents 60 which may react (e.g., cell lysis) with sample contained in the swab tip 30. As shown in FIG. 6, the reaction tube 51 may be covered by a cap 52 in order to seal the head portion 2 and reagents 60 within the tube 51. In other embodiments, as described previously, the tube 51 may have a frangible seal to protect the swab 10 (which may include just the head portion 2 or the unbroken swab 10 with a handle 20) and reagents 60 in the tube 51.

As shown in FIG. 6, the reaction tube 51 may include a width W5. In some embodiments, width W5 may be greater than any of the widths W1, W2, W3, and/or W4, such that the swab 10 may be inserted into the reaction tube 51. In some embodiments, any width of the head portion 2 may be less than the width W5, while the width W1 of the handle 20 (see FIG. 4) may be greater than the width W5. Width W5 may be suitably sized to allow a user to tilt the swab 10 against a sidewall of the tube 51 to help break the breakable portion 25 without spilling the reagent 60. In other words, width W5 may be suitably sized in accordance with the head portion 2 height and width to allow the breakable portion to break without the user needing to contact the swab tip 30. Of course, embodiments where the breakable portion 25 is broken without the aid of the tube 51 sidewall are also contemplated, such that the width W5 may be significantly greater than any of the widths W1, W2, W3, and/or W4. Accordingly, the reaction tube 51 width W5 may be any suitable size, as the present disclosure is not so limited.

As shown in FIG. 5, in some embodiments, the amplification reservoir 54 may include lyophilized amplification reagents (e.g., a lyophilized amplification bead), as described herein. The amplification reagents may be RT/RPA or LAMP. The reagents may be hydrated either by contact with the lysate, or by the addition of a dilution buffer from the optional dilution buffer reservoir 53. As described above, the dilution buffer may be released from its reservoir 53 and into the amplification reservoir 54 by puncturing a film and then transporting the buffer through its designated channel 59 with the pumping tool 58.

In some embodiments, the amplification reservoir 54 or any other reservoir may be heated to 37° C. for amplification. After amplification, the resulting fluid (which may be sample reacted with any combination of reagents described herein) may be transported to the readout element 56 using a unique pump channel 59. The readout element 56, in some embodiments, uses angled pocket geometry. After this process, the assay may be completed, and the user may determine the results using any of the methods described herein (e.g., comparing the results to a key, using a mobile app, etc.). The cartridge 5 may also include an air expansion reservoir 55 which maintains the atmospheric pressure in the amplification reservoir 54 and readout element 56 area, while maintaining a hermetic seal to prevent contamination. A thin heat-seal plastic film layer behaves as a chemical barrier and as a pressure diaphragm. In some embodiments, seal plate 57 may be formed of FR4/G10 (or any other inexpensive semiconductor material), and may be attached to a main-body cartridge 50 by fasteners (e.g., glue, screws). If a dilution buffer is not needed, its corresponding channel on the seal plate may be blocked off and is not functional (e.g., to prevent user error).

In some embodiments, the main-body cartridge 50 may include a printed circuit board (PCB) heater and battery. The PCB heater, in some embodiments, comprises a bonded PCB with a microcontroller, thermistors, and resistive heaters located below the lysis reservoir 51 and amplification reservoir 54. In this way, the heater may be able to maintain the required temperatures for lysis and amplification. The heater, in some embodiments, comprises a USB mini power connector. In some embodiments, the heater may be pre-programmed to lyse and to amplify the sample (e.g., it runs a lysis protocol and an amplification protocol). A lysis protocol is one in which lysis of the sample occurs, such that the sample can be further processed, as described herein. An amplification protocol may be one in which amplification of the sample occurs, such that the sample can be analyzed (i.e., the sample may be sufficiently amplified to be detected on the readout element (e.g., lateral flow assay strip).

In some embodiments, the microcontroller (or any other component or combination of components) of the main-body cartridge 50 may be able to communicate with an external network or system. For example, the heater may be controlled by a mobile application. In other embodiments, a companion mobile application may alert a user as to when the heating protocol (e.g., lysis, amplification) is complete. In some embodiments, the mobile application may be able to store information regarding the temperatures used during the processing steps. In a further embodiment, the cartridge 5 may be connected to the mobile application via a wired connection or through a wireless connection 7, as shown in FIG. 5. In some embodiments, the wireless (e.g., Bluetooth®) connection allows the mobile application to store all of the information from the heating device. In some embodiments, the wireless, e.g., Bluetooth®, connection allows a user to select the different heating/cooling protocol as needed. In some embodiments, the heating/cooling protocol may be selected remotely or automatically (e.g., not by the immediate user) upon detection of the device and/or test type.

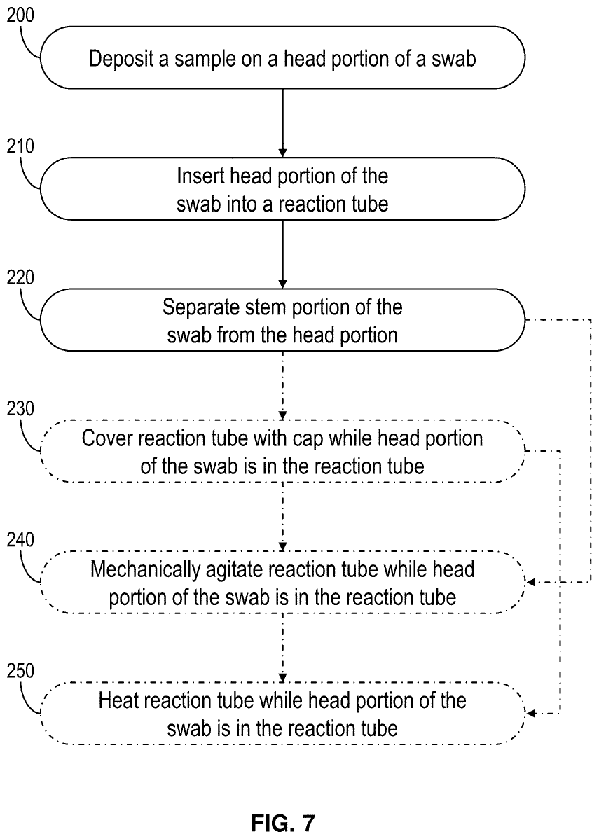

FIG. 7 shows, according to some embodiment, a flow chart for a method of collecting and processing a sample. In block 200, a sample is deposited on a head portion (which may include a swab tip in some embodiments). This sample deposition (or sample collection) process is depicted by FIG. 1. In block 210, the head portion of the swab, which may contain the sample, is inserted into a reaction tube. The reaction tube may be part of a diagnostic device or any other suitable sample-receiving system. In block 220, the stem portion of the swab may be separated or detached from the head portion. In some embodiments, the head portion may remain in the reaction tube when the stem portion is detached. As described in further detail above, the head portion may be separated from the stem portion of the swab at a breakable portion, which may be more susceptible to breakage than the stem portion and/or the head portion. FIG. 7 also depicts an optional block 230, in which the reaction tube may be covered (e.g., with a cap, as depicted in FIG. 6) after separation of the head portion and stem portion. In some embodiments, the reaction tube may remain uncovered during the diagnostic test. In other embodiments, the reaction tube may include a seal to reduce the risk of contamination of the sample and/or reagents. In another optional block, block 240, the reaction tube may be mechanically agitated while the head portion is located in the reaction tube. The reaction tube may be mechanically agitated before separation of the stem portion, after separation of the stem portion, or at any other point in the diagnostic test. In some embodiments, the reaction tube may not be mechanically agitated. In some embodiments, as depicted in FIG. 7, the reaction tube may be mechanically agitated without the step of block 230. In yet another optional block, block 250, the reaction tube may be heated while the head portion is located in the reaction tube. The reaction tube may be heated before separation of the stem portion, after separation of the stem portion, or at any other point in the diagnostic test. In some embodiments, the reaction tube may not be heated. In some embodiments, as depicted in FIG. 7, the reaction tube may be heated without the step of block 230. As described previously, heating and/or mechanically agitating the reaction tube may increase the volume of sample released from the swab tip. It should be appreciated that the optional steps (blocks 230, 240, and 250) and any other processing steps may be conducted in any suitable order.

Other embodiments of the cartridge are also contemplated. For example, in some embodiments, a cartridge may include blister packs. In some embodiments, the various reagents for the diagnostic test are stored in lab on chip reagent blister packs. The blister packs can be, for example, seal blister packs (frangible seal blister packs), wherein the reagents are each delivered in a controlled manner using differential seal technology. In some embodiments, the blister packs are multi-chamber blister packs; that is, the blister pack may store multiple components (both liquid and solid) in different chambers. For example, lyophilized reagents can be stored in individual chambers, while the buffers or solutions necessary to resuspend the lyophilized reagents can each be stored in separate chambers, separated by a frangible seal.

Of course, the cartridge 5 depicted in FIG. 5 and described herein is an exemplary embodiment of the diagnostic device used with the swab 10. It should be appreciated that the swab 10 may be used with any suitable diagnostic device or other sample-receiving system, and that the current disclosure is not limited by the auxiliary equipment which may be used with the swab 10.

### Kits

Any of the devices described herein in combination with the breakable swab may be formulated as a kit. As used herein, a “kit” comprises a package or an assembly including one or more of the test compositions of the invention. Any one of the kits provided herein may comprise any number of breakable swabs, reaction tubes, wells, chambers, or other vessels. Each of the components of the kit (e.g., reagents) may be provided in liquid form (e.g., in solution) or in solid form (e.g., a dried powder, lyophilized). Several exemplary kits and methods of using them are described below.

In one embodiment, the kit comprises a sterile breakable swab, a cap, an amplification cap, a heating device, and a readout element. After taking a nasal or cheek swab sample, the breakable swab may be inserted into a reaction tube containing a volume of rehydration buffer (e.g., 500 uL of PBS) and mixed around for 10 seconds. The breakable swab may then be separated, leaving a sample-containing swab tip within the tube, and a cap may be added to the reaction tube. The tube may then be placed in a heating device (such as a USB-powered heating device) at 37° C. for three minutes, and then ramped up to 65° C. and held there for 10 minutes. The temperature may then be reduced back down to 37° C. The cap may be removed and replaced by an amplification cap. In some embodiments, the amplification cap comprises a foil seal top that may be punctured or removed when the cap is placed on the tube, exposing the lyophilization bead to the solution. The amplification cap comprises a reverse transcriptase and RPA lyophilized bead. In other embodiments, the amplification cap comprises a lyophilized version of LAMP reagents. The reaction tube, now comprising the amplification cap, may then be inverted until the bead dissolves. Then, the reaction tube may be heated to 37° C. for 15 minutes for amplification, for example, using a USB-powered heating device. Then, in some embodiments, the reaction tube may be added to a readout element, and the readout element then runs the same through a lateral flow test, and the results of the test (e.g., positive or negative for the viral illness(es) screened) using ARUCO markers, are reported in a mobile app. In some embodiments, the readout element dilutes the sample, if needed, prior to running the lateral flow test. In other embodiments, dilution is not necessary because an alternative probe, such as a dual-hapten probe described herein, has been used.

In another embodiment, the kit comprises a sterile breakable swab, a blister cap, a heating device, and a readout element. After taking a nasal or cheek swab sample, the breakable swab may be inserted into a reaction tube containing a volume of rehydration buffer (e.g., 500 uL of PBS) and mixed around for 10 seconds. The sample-containing swab tip of the breakable swab may then be separated from the stem portion of the swab, and the blister cap may be added to the reaction tube. The tube may then be placed in the heating device (such as a USB-powered heating device) at 37° C. for three minutes, and then ramped up to 65° C. and held there for 10 minutes. The temperature may then be reduced back down to 37° C. The blister cap may then be pushed, so that it releases its cargo, in this case, an amplification pellet comprising the lyophilized reagents necessary for amplification of the sample. The reaction tube, now comprising the amplification cap, may then be inverted until the bead dissolves. Then, the reaction tube may be heated to 37° C. for 15 minutes for amplification, for example, using a USB-powered heating device. Then, in some embodiments, the reaction tube may be added to a readout element, and the readout element then runs the same through a lateral flow test, and the results of the test (e.g., positive or negative for the viral illness(es) screened) are determined with ARUCO markers, and are reported in a mobile app. In some embodiments, the readout element dilutes the sample, if needed, prior to running the lateral flow test. In other embodiments, dilution is not necessary because an alternative probe, such as a dual-hapten probe described herein, has been used. The temperatures and times listed are exemplary and may differ from those described.

### Diagnostic Test Applications

Diagnostic devices, systems, and methods described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents, buffers, diluents, or any other appropriate materials may be contained within fluid containers (e.g., reservoirs) of the device. In this way, users may have limited exposure to any potentially harmful chemicals, and the fluids and/or materials necessary for the diagnostic test may be protected from contamination (either from surrounding gases/fluids or from cross-contamination within the device) until operation.

Diagnostic devices, systems, and methods described herein are also highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers. In such cases, untrained consumers may be able to self-administer the diagnostic test (or administer the test to friends and family members) in their own homes (or any other location of their choosing). In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist).

In some embodiments, diagnostic devices described herein are relatively small. In certain cases, for example, a diagnostic device (such as an exemplary cartridge 5 shown in FIG. 5) may be approximately the size of an adult hand. Thus, unlike diagnostic tests that require bulky equipment, diagnostic devices and systems described herein may be easily transported and/or easily stored in homes and businesses. In some embodiments, the diagnostic devices and systems may be relatively inexpensive. Since no expensive laboratory equipment (e.g., a thermocycler) is required, diagnostic devices, systems, and methods described herein may be more cost effective than known diagnostic tests.

In some embodiments, the breakable swab described herein may be shelf stable for a relatively long period of time. In certain embodiments, for example, the breakable swab may be stored at room temperature (e.g., 20° C. to 25° C.) for a relatively long period of time (e.g., at least 1 month, at least 3 months, at least 6 months, at least 9 months, at least 1 year, at least 5 years, at least 10 years). In certain embodiments, the breakable swab may be stored across a range of temperatures (e.g., 0° C. to 20° C., 0° C. to 37° C., 0° C. to 60° C., 0° C. to 90° C., 20° C. to 37° C., 20° C. to 60° C., 20° C. to 90° C., 37° C. to 60° C., 37° C. to 90° C., 60° C. to 90° C.) for a relatively long period of time (e.g., at least 1 month, at least 3 months, at least 6 months, at least 9 months, at least 1 year, at least 5 years, at least 10 years).

### Target Nucleic Acid Sequences

The diagnostic devices, systems, and methods described herein may be used to detect the presence or absence of any target nucleic acid sequence (e.g., from any pathogen of interest) or multiple target nucleic acid sequences. Target nucleic acid sequences may be associated with a variety of diseases or disorders. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are used to diagnose at least one disease or disorder caused by a virus, bacteria, fungus, protozoan, parasite, and/or cancer cell. Of course, a diagnostic test according to exemplary embodiments described herein may be employed to detect any desired target nucleic acid sequence, as the present disclosure is not so limited.

### Nucleic Acid Amplification

Following lysis, one or more target nucleic acids (e.g., a nucleic acid of a target pathogen) may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification. In some embodiments, reverse transcription is performed by exposing lysate to one or more reverse transcription reagents. In certain instances, the one or more reverse transcription reagents comprise a reverse transcriptase, a DNA-dependent polymerase, and/or a ribonuclease (RNase). In some embodiments, DNA may be amplified according to any nucleic acid amplification method known in the art.

### Loop-Mediated Isothermal Amplification (LAMP)

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence.

### RPA

In some embodiments, the nucleic acid amplification reagents are RPA reagents. RPA generally refers to a method of amplifying a target nucleic acid using a recombinase, a single-stranded DNA binding protein, and a strand-displacing polymerase.

In any of the embodiments described herein, the LAMP reagents or the RPA reagents may be lyophilized and formulated as one or more beads. These beads are referred to herein as “amplification beads” or “amplification pellets.” As described herein, the amplification beads may be added to any of the tests provided herein, for example, as part of a cap/lid designed to release the amplification bead(s) into solution after the sample has been mixed into a buffer or as part of a blister pack in a lid, such that the amplification bead may be contacted with the sample.

### Nicking Enzyme Amplification Reaction (NEAR)

In some embodiments, amplification of one or more target nucleic acids is accomplished through the use of a nicking enzyme amplification reaction (NEAR) reaction. NEAR generally refers to a method for amplifying a target nucleic acid using a nicking endonuclease and a strand displacing DNA polymerase. In some cases, NEAR may allow for amplification of very small amplicons.

### Molecular Switches

As described herein, a sample undergoes lysis and amplification prior to detection. In certain embodiments, one or more (and, in some cases, all) of the reagents necessary for lysis and/or amplification are present in a single pellet or tablet. In some embodiments, a pellet or tablet may comprise two or more enzymes, and it may be necessary for the enzymes to be activated in a particular order. Therefore, in some embodiments, the enzyme tablet further comprises one or more molecular switches. Molecular switches, as described herein, are molecules that, in response to certain conditions, reversibly switch between two or more stable states. In some embodiments, the condition that causes the molecular switch to change its configuration is pH, light, temperature, an electric current, microenvironment, or the presence of ions and other ligands. In one embodiment, the condition is heat. In some embodiments, the molecular switches described herein are aptamers. Aptamers generally refer to oligonucleotides or peptides that bind to specific target molecules (e.g., the enzymes described herein). The aptamers, upon exposure to heat or other conditions, may dissociate from the enzymes. With the use of molecular switches, the processes described herein (e.g., lysis, decontamination, reverse transcription, and amplification) may be performed in a single test tube with a single enzymatic tablet.

### Detection/Diagnosis

In some embodiments, amplified nucleic acids (i.e., amplicons) may be detected and communicated to a user using any suitable method or combination of methods. In some embodiments, one or more target nucleic acid sequences are detected using a lateral flow assay strip (e.g., disposed in a diagnostic device). In some embodiments, a fluidic sample (e.g., comprising a particle-amplicon conjugate) may be configured to flow through a region of the lateral flow assay strip (e.g., a test pad) comprising one or more test lines. In some embodiments, a first test line comprises a capture reagent (e.g., an immobilized antibody) configured to detect a first target nucleic acid and an opaque marking may appear if the target nucleic acid is present in the fluidic sample. The marking may have any suitable shape or pattern (e.g., one or more straight lines, curved lines, dots, squares, check marks, x marks). In certain embodiments, the lateral flow assay strip comprises one or more additional test lines. In some instances, each test line of the lateral flow assay strip may be configured to detect a different target nucleic acid. In certain embodiments, the region (e.g., the test pad) of the lateral flow assay strip generating an opaque marking further comprises one or more control lines to indicate that a human (or animal) sample was successfully collected, nucleic acids from the sample were amplified, and that amplicons were transported through the lateral flow assay strip.

In some embodiments, the detection may be performed through a colorimetric assay, that is, a chromogenic reaction is performed. For example, the processed sample may be exposed to reagent that undergoes a color change when bound to the viral DNA, such as with an enzyme-linked immunoassay. In some embodiments, the assay further comprises a stop reagent, such as sulfonic acid. That is, when the processed sample may be mixed with the reagents, the solution turns a specific color (e.g., red) if the pathogenic DNA is present, and the sample may be positive for the virus. If the solution turns a different color (e.g., green), the pathogenic DNA is not present and the sample may be negative for the virus. As described above, the colorimetric assay may be a colorimetric LAMP assay; that is, the LAMP reagents react in the presence or absence of a target sequence (e.g., from COVID-19) to turn one of two colors.

The diagnostic test used with the breakable swab described herein, in some embodiments, may be used to diagnose at least one disease or disorder caused by a pathogen, as described below. In some embodiments, the tests may be designed so that a user can differentiate between one or more diseases or disorders (e.g., a lateral flow test comprises more than one test line). In one embodiment, the lateral flow test comprises a test line for SARS-CoV-2 and a test line for an influenza virus (e.g., Type A or Type B). In another embodiment, the lateral flow test comprises a test line for SARS-CoV-2, influenza Type A, and influenza Type B. In further embodiments, the test may be used to differentiate between viral and bacterial infections.

In some embodiments, the tests are used to diagnose or detect a viral illness, such as a respiratory illness, including, but not limited to, those caused by coronaviruses, influenza viruses, rhinoviruses, parainfluenza viruses (e.g., parainfluenza 1-4), enteroviruses, adenoviruses, respiratory syncytial viruses, and metapneumoviruses, or any other suitable illness caused by any viral infectious agents or combination of agents. In some embodiments, the tests may be used to diagnose one or more bacterial infections. The bacterium described herein can be a Gram-positive bacterium or a Gram-negative bacterium. In some embodiments, the tests described herein may be used to diagnose one or more fungal infections caused by fungal pathogens. In some embodiments, the tests described herein may be used to diagnose one or more protozoan infections caused by protozoan pathogens. In some embodiments, the tests described herein may be used to diagnose one or more parasitic infections caused by parasitic pathogens. The test may also be used to diagnose any number of pathogenic animal diseases. The tests described herein may also be used to test water or food for contaminants (e.g., bacteria). In some embodiments, the tests described herein may also be used for soil analysis. The tests may also be used as diagnostics for various cancers.

### INSTRUCTIONS & SOFTWARE

In some embodiments, a diagnostic system used with the breakable swab described herein may include instructions for using a diagnostic device and/or otherwise performing a diagnostic test method. The instructions may include instructions for the use, assembly, and/or storage of the diagnostic device and any other components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications). In some embodiments, the instructions are provided as part of a software-based application. In certain cases, the application can be downloaded to a smartphone or device, and then guides a user through steps to use the diagnostic device.

In some embodiments, a software-based application may be connected (e.g., via a wired or wireless connection) to one or more components of a diagnostic system (e.g., through connection 7 to cartridge 5, as shown in FIG. 5).

In some embodiments, a diagnostic system comprises or is associated with software to read and/or analyze test results. In some embodiments, a device (e.g., a camera, a smartphone) may be used to generate an image of a test result (e.g., one or more lines detectable on a lateral flow assay strip). In some embodiments, a user may use an electronic device (e.g., a smartphone, a tablet, a camera) to acquire an image of the visible portion of the lateral flow assay strip. In some embodiments, software running on the electronic device may be used to analyze the image (e.g., by comparing any lines or other markings that appear on the lateral flow assay strip with known patterns of markings). A machine vision software application may be employed to read the uploaded or entered test reading, and automatically provide a positive or negative test result. The result may be communicated directly to a user or to a medical professional. In some cases, the test result may be further communicated to a remote database server. In some embodiments, the remote database server stores test results as well as user information such as at least one of name, social security number, date of birth, address, phone number, email address, medical history, and medications. In other embodiments, the mobile application sends this information (e.g., an image of the resultant lateral flow test strip) to a secure, HIPAA-compliant, cloud-based software infrastructure. This software infrastructure then facilitates simple, fast, and scalable reporting to the federal and state health agencies.

In some embodiments, the database may generate a code based on the user's results (e.g., positive or negative for the viral illness). After a successful test, the code is available in the application. In some embodiments, the code may be read by a bar code scanner or other security detection device. If the user is negative for the viral illness and has a negative code, the security system will recognize the code and permit entry. In other embodiments, if the user is positive for the viral illness and has a positive code, the security system will recognize the code and deny entry.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

What is claimed is:

1. A swab for sample collection, the swab comprising:
an elongated body including a first end and a second end;
a head portion extending from the first end to a breakable portion; and
a stem portion extending from the second end to the breakable portion, the breakable portion positioned along the elongated body between the head portion and the stem portion,
wherein the stem portion is configured to be detached from the head portion at the breakable portion.

2. The swab of claim 1, wherein the head portion comprises a swab tip configured to hold a sample, and wherein the stem portion comprises a handle configured to be handled by a user.

3. The swab of claim 1, wherein the head portion is configured to be inserted into a nasal cavity or an oral cavity of a subject.

4. The swab of claim 1, wherein a first cross-sectional width of the stem portion is greater than a second cross-sectional width of the breakable portion, the first cross-sectional width and the second cross-sectional width measured across a central axis extending along the elongated body.

5. The swab of claim 1, wherein the stem portion is detached from the head portion after insertion of the head portion into a reaction tube.

6. The swab of claim 2, wherein the head portion is inserted into a reaction tube, and wherein the reaction tube including the head portion and the sample is heated above 37° C.

7. The swab of claim 2, wherein the head portion is inserted into a reaction tube, and wherein the reaction tube including the head portion and the sample is mechanically agitated.

8. The swab of claim 1, wherein the swab is used with a rapid test that detects presence or absence of at least one pathogen selected from COVID-19, an influenza virus, or a target nucleic acid.

9. The swab of claim 8, wherein the rapid test is an isothermal test including a lateral flow strip and result readout element.

10. The swab of claim 1, wherein the breakable portion includes one or more features configured to lower a resilience of the breakable portion with respect to a resilience of the stem portion.

11. A swab for sample collection, the swab comprising:
a head portion extending from a first end of the swab to a breakable portion of the swab; and
a stem portion extending from a second end of the swab to the breakable portion,
wherein the stem portion is configured to be detached from the head portion at the breakable portion, and wherein a cross-sectional width of the breakable portion measured along a central axis of the swab is less than a cross-sectional width of the head portion.

12. The swab of claim 11, wherein the cross-sectional width of the breakable portion is less than a cross-sectional width of the stem portion.

13. The swab of claim 11, wherein the head portion comprises a swab tip configured to hold a sample, and wherein the stem portion comprises a handle configured to be handled by a user.

14. The swab of claim 11, wherein the breakable portion includes one or more features configured to lower a resilience of the breakable portion with respect to a resilience of the stem portion.

15. The swab of claim 13, wherein the sample is a mucus sample or a saliva sample.

16. The swab of claim 11, wherein the stem portion is detached from the head portion after insertion of the head portion into a reaction tube.

17. The swab of claim 13, wherein the head portion is inserted into a reaction tube, and wherein the reaction tube including the head portion and the sample is heated above 37° C.

18. A method of processing a sample, the method comprising:
inserting a head portion of a swab into a nasal or oral cavity of a subject, the head portion positioned at one end of the swab, the swab including a stem portion positioned at an opposing end of the swab from the head portion;
inserting the head portion of the swab into a reaction tube; and
separating the stem portion from the head portion.

19. The method of claim 18, further comprising mechanically agitating the reaction tube including the head portion of the swab.

20. The method of claim 18, further comprising heating the reaction tube including the head portion of the swab above 37° C.
